(19) United States
(12) Patent Application Publication
(10) Pub. No.: US 2010/0189697 A1
(43) Pub. Date: Jul. 29, 2010

(54) CARDIAC-SPECIFIC PROGENITOR CELLS

(76) Inventors: Andre Terzic, Rochester, MN (US); Atta Behfar, Rochester, MN (US); Timothy J. Nelson, Rochester, MN (US)

Correspondence Address: FISH & RICHARDSON P.C., PO BOX 1022, MINNEAPOLIS, MN, 55440-1022, US

(21) Appl. No.: 12/530,165

(22) PCT Filed: Mar. 7, 2008
(86) PCT No.: PCT/US08/56248

§ 371 Date: Mar. 5, 2010

Related U.S. Application Data
(60) Provisional application No. 60/905,513, filed on Mar. 7, 2007.

(51) Int. Cl. A61K 35/34; A61P 9/00; C12N 5/071; C12N 5/0735

(52) U.S. Cl. 424/93.7; 435/325; 435/375

(57) ABSTRACT

This document provides methods and materials related to treating cardiovascular tissue (e.g., heart tissue or vascular tissue). For example, stem cells (e.g., CXCR4+/Flk-1+ stem cells), compositions containing stem cells, methods for obtaining stem cells, and methods for repairing cardiovascular tissue are provided.

### STATEMENT AS TO FEDERALLY SPONSORED RESEARCH

This invention was made with government support under grant HLO83439 awarded by National Institutes of Health. The government has certain rights in the invention.

### BACKGROUND

1. Technical Field

This document relates to methods and materials involved in cardiac-specific progenitor cells and treating cardiovascular tissue.

2. Background Information

Stem cells, with their unique balance between pluripotent self-renewal and tissue-specific differentiation, are the focal point for applications of cardiac regenerative medicine (Srivastava and Ivey, (2006) Nature, 441, 1097-1099 and Foley and Mercola, (2004) Trends Cardiovasc. Med., 14, 121-125). Stem cell transplantation in the setting of acute ischemic injury has progressed from pre-clinical evidence of cell-based repair and functional recovery to phase II clinical trials (Sanchez et al., (2006) Nat. Clin. Pract. Cardiovasc. Med., 3 Suppl. 1, S138-151).

### SUMMARY

This document provides methods and materials related to treating cardiovascular tissue (e.g., heart tissue or vascular tissue). For example, this document provides stem cells (e.g., CXCR4+/Flk-1+ stem cells), compositions containing stem cells, methods for obtaining stem cells, and methods for repairing cardiovascular tissue. The stem cells and compositions containing stem cells provided herein can be used to repair cardiovascular tissue. Such stem cells can allow clinicians to treat, for example, myocardial injury since the stem cells can have the ability to differentiate into cardiomyocytes. In some cases, such stem cells and compositions containing stem cells can be used to repair cardiovascular tissue without producing tumors within the tissue. The methods and materials provided herein can allow medical professionals to produce large amounts of stem cells that can express particular markers and can be used to repair cardiovascular tissue.

In general, one aspect of this document features an enriched population of stem cells comprising a CXCR4 polypeptide and an Flk-1 polypeptide, wherein the stem cells are capable of differentiating into cells that express Mef2C, GATA-4, Myocardin, and Nkx2.5 polypeptides. The stem cells can be human stem cells. The stem cells can be embryonic stem cells.

In another aspect, this document features a method for obtaining cardiac-specific progenitor cells. The method comprises, or consists essentially of, obtaining an enriched population of CXCR4+/Flk-1+ stem cells capable of differentiating into cells that express Mef2C, GATA-4, Myocardin, and Nkx2.5 polypeptides from a population of stem cells. The CXCR4+/Flk-1+ stem cells can be CXCR4+/Flk-1+ embryonic stem cells. The population of stem cells can comprise CXCR4+/Flk-1+ stem cells and CXCR4−/Flk-1− stem cells. The CXCR4+/Flk-1+ stem cells can be human cells. The obtaining step can comprise using anti-CXCR4 antibodies, anti-Flk-1 antibodies, and a cell sorter to obtain the enriched population of CXCR4+/Flk-1+ stem cells. The obtaining step can comprise using anti-CXCR4 antibodies, anti-Flk-1 antibodies, and panning to obtain the enriched population of CXCR4+/Flk-1+ stem cells.

In another aspect, this document features a method for providing heart tissue with cardiomyocytes. The method comprises, or consist essentially of, administering, to the heart tissue, an enriched population of stem cells comprising a CXCR4 polypeptide and an Flk-1 polypeptide, wherein the stem cells are capable of differentiating into cells that express Mef2C, GATA-4, Myocardin, and Nkx2.5 polypeptides. The cardiomyocytes can be human cardiomyocytes. The stem cells can be embryonic stem cells. The heart tissue can be ischemic heart tissue or heart tissue that has suffered from myocardial infarction.

In another aspect, this document features a method for providing heart tissue with cardiomyocytes. The method comprises, or consist essentially of, administering, to the heart tissue, cardiomyocytes obtained from an enriched population of stem cells comprising a CXCR4 polypeptide and an Flk-1 polypeptide, wherein the stem cells are capable of differentiating into cells that express Mef2C, GATA-4, Myocardin, and Nkx2.5 polypeptides. The cardiomyocytes can be human cardiomyocytes. The stem cells can be embryonic stem cells.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 depicts a cytokine gene cluster prioritized from a hierarchically organized transcriptome of embryonic stem cells at the cardiopoietic stage. Differentiating embryonic stem cells at four distinct stages of cardiogenesis, i.e.—undifferentiated embryonic stem cells developmentally restrained by LIF (ES), released from LIF (ES′), cardiopoietic stem cells (CP), and sarcomere-rich cardiomyocytes (CM), were collected and analyzed with microarray gene expression assays. (A). Restricted expression profiling demonstrated dynamic transcriptional changes from ES to CP, delimiting 11,272 genes. Numbers to the right of the scale indicate fold change. (B). Expression heatmap representation of 736 membrane restricted genes extracted from thresholded CP transcripts were hierarchically clustered, displaying reproducible molecular fingerprints for three biologically distinct samples. (C). Data mining of 306 up-regulated, membrane-associated CP transcripts revealed prominence of a hierarchically arranged cytokine cluster comprised of 11 genes. Inset scale represents fold change.

FIG. 2 depicts CXCR4 chemokine receptor resolved within the cardiopoietic cytokine cluster. (A). Analysis of overlapping transcripts among the KEGG pathway designated cytokine/chemokine family, the GO Consortium classification of genes integral to membrane, and the cardiopoietic transcriptome yielded 16 common genes. (B). Logical set exclusion identified 69% (11/16) of overlapping genes both Venn-restricted and up-regulated in the CP stage (filled circles). (C). Analysis of bioinformatically qualified genes (filled circles) integrates into a functional network with relationships curated by the Ingenuity Pathways Knowledge Base (IPKB). The contextualized cytokine cluster integrates the CXCR4 chemokine receptor (large filled circle) that directly couples the extracellular signaling milieu with the intranuclear transcriptional machinery.

FIG. 3 indicates that CXCR4 is a chemokine receptor family member induced in differentiating embryonic stem cells prior to cardiac differentiation. (A). Differentiating embryonic stem cells collected at the four distinct stages of cardiogenesis: ES, ES′, CP, and CM. Expression levels for all 26 genes within the chemokine receptor families were analyzed using the microarray data from three independent experiments run in triplicates. Five genes (CXCR4, CXCR3, CXCR6, and GPR27) had detectable expression, with CXCR4 demonstrating a 4-fold induction in cardiopoietic embryonic stem cells compared to ES cells. (*p<0.05) RT-PCR confirmed the induction of CXCR4 relative to GAPDH expression in cardiopoietic cells compared to undifferentiated embryonic stem cells (Inset). In the graphs, CXCR4 is the first bar, CXCR3 is the second bar, CXCR6 is the third bar, and Gpr27 is the fourth bar. (B). Differentiating embryonic stem cells spanning the cardiopoietic window demonstrated the transition from day 5 embryonic stem cell-derived progeny devoid of cardiac Mef2c or α-actinin to day 7 cardiopoietic stem cells with nuclear Mef2c expression (green), followed by maturation to sacromere-rich cardiomyocytes expressing α-actinin (red) at day 9. Consistent with the maturation of the cardiac phenotype, spontaneously contracting cells were observed at Day 8. All nuclei were stained with DAPI (blue). (C). Embryonic stem cells were harvested every 48 hours between day 0 and day 9 for mRNA extraction and gene expression analysis using Taqman RT-PCR. Pluripotent markers, Oct4 and FGF4, were decreased by day 3 of differentiation with particularly significant reduction at day 5. CXCR4 expression conversely surged by day 5 and remained significantly elevated compared to undifferentiated embryonic stem cells. CXCR4 induction preceded canonical cardiac markers, Myocardin, Mef2c and Nkx2.5 initially expressed at day 7 of differentiation.

FIG. 4 indicates CXCR4 cell surface induction coincides with mesoderm specification during in vitro differentiation of embryonic stem cells. (A). Differentiating embryonic stem cells in embryoid bodies at day 3 and day 5 were dissociated into single cells and stained for CXCR4 protein expression on living cells. Flow sorting analysis revealed that only 3% of total cells expressed detectable levels of CXCR4 at day 3 (D3). The number of cells expressing CXCR4 increased to 50% by day 5 (D %) in embryoid bodies. (B). Differentiating embryoid bodies were harvested at 48 hour intervals, and lineage specific gene expression was analyzed with Taqman RT-PCR. Temporal expression dynamics of endoderm genes Sox7 and Sox17 as well as mesoderm genes Gsc and Lhx1 demonstrated peaked expression levels between days 5 and 7, representing concomitant lineages. (C). Flk-1 (VEGF-receptor 2), a specific marker of primitive mesoderm, also demonstrated an expression profile that was induced at day 5 and peaked at day 7, coinciding with the induction kinetics of CXCR4. In the bar graph, CXCR4 is the first bar and Flk-1 is the second bar.

FIG. 5 indicates that a CXCR4⁻/Flk-1⁻ sub-population can be distinguished from CXCR4⁻/Flk-1⁻ counterparts by flow cytometry. Embryonic stem cells were freshly prepared from embryoid bodies at day 3, 5, and 7 (D3, D5, and D7) for flow sorting analysis using both CXCR4 and Flk-1 antibodies. CXCR4 and Flk-1 cell surface protein expression was largely absent from day 3 cells with less than 3% and 1% of cells expressing each marker within the total population, respectively. Both the CXCR4 and Flk-1 biomarkers were induced at day 5, consistent with temporal gene expression profile. The double positive CXCR4⁺/Flk-1⁺ sub-population surged transiently to 32% of total population at day 5. The double positive CXCR4⁺/Flk-1⁺ subpopulation surged transiently to 32% of total population at day 5. The CXCR4⁻/Flk-1⁻ (1^{st} bar in each graph), CXCR4⁺/Flk-1⁺ (2^{nd} bar in each graph) and CXCR4⁺/Flk-1⁺ (3^{rd} bar in each graph) subpopulations were collected at day 5 for gene expression analysis (B-C). (B): Both CXCR4 and Flk-1 were highly expressed in CXCR4⁺/Flk-1⁺ cells, while CXCR4⁻/Flk-1⁺ and CXCR4⁻/Flk-1⁻ subpopulations expressed, respectively, only Flk-1 at high levels or did not express either marker indicating cell sorting quality. The pluripotency marker Oct-4 was highly expressed in CXCR4⁻/Flk-1⁻ cells compared to either CXCR4⁻/Flk-1⁺ or CXCR4⁺/Flk-1⁺ subpopulations. (C): CXCR4⁺/Flk-1⁺ was characterized by high expression of mesoderm markers, Lhx and Gsc, and genes associated with the primary heart field, GATA-4 and Tbx5, and with low expression of smooth muscle and vascular endothelial markers, Myh11 and CD31, compared to CXCR4⁻/Flk-1⁻ or CXCR4⁻/Flk-1⁺ cells. Star indicates P<0.05 throughout.

FIG. 6 indicates that biomarker-sorted embryonic stem cells select progenitors with a pre-cardiac transcriptome. (A): Post-sort analysis of collected CXCR4⁻/Flk-1⁻ (−/−) and CXCR4⁺/Flk-1⁺ (+/+) cells demonstrated >90% purity in respective subpopulations. (B): While CXCR4⁻/Flk-1⁻ cells demonstrated poor immunocytochemical labeling for Mesp-1 (left), the CXCR4⁺/Flk-1⁺ subpopulation expressed high levels of this pre-cardiac mesoderm marker. (C): Volcano plot analysis segregates differentially expressed genes in CXCR4⁺/Flk-1⁺ compared to CXCR4⁻/Flk-1⁻ subpopulations. Dark grey identifies genes significantly changing >1.2 fold, while light grey marks all sub-threshold genes. Inset: Proportion of interrogated transcriptome showing no change (97%) and significant change (3%) between the two subpopulations. (D): Left, ontological analysis of circumscribed significantly changing genes yield overrepresented functional families, listed in order of priority. Right, Contribution of CXCR4 and Flk-1 in respective functional systems identifies the combined presence of both markers for “Cardiovascular Development”. The P-values are provided with exponential notation. Dashes indicate P>0.05. (E): Curated network construction integrating genes involved in “Cardiovascular Development”. CXCR4 and Flk-1 are outlined, with light grey and dark grey node colors indicating up- and down-regulated genes, respectively.

FIG. 7 indicates that CXCR4⁺/Flk-1⁺ cells differentiate into functional cardiac tissue. (A-C): Cells sorted from day-5 old embryoid bodies were differentiated in a monolayer for 4 days. In contrast to CXCR4⁻/Flk-1⁻ progeny (left panels), CXCR4⁺/Flk-1⁺ counterparts (right panels) demonstrated positive expression of cardiac a-actinin (red; A-B) with nuclear localization of cardiac transcription factors Mef2c (green; A) and Nkx2.5 (green; B) consistent with cardiac phenotype. Conversely, CXCR4⁻/Flk-1⁻ (left panel) in contrast to CXCR4⁺/Flk-1⁺ (right panel) progeny expressed the endoderm marker alpha-fetoprotein AFP (green; C). Cell nuclei were counterstained with DAPI (blue; A-C). Scale bar, 20 μm (A-C). (D): Gene expression analysis using unsorted cells as baseline (100% expression) revealed CXCR4⁻/Flk⁻ cells enriched for pluripotency (Oct4, Fgf4), neuroectoderm and ectoderm (Sox1, Sox2). In contrast, CXCR4⁺/Flk⁺ cells were enriched for markers of cardiac differentiation with Mef2c expression increased by ≧1200% and Nkx2.5, GATA-4, and myocardin increased by ≧300%. In the bar graph, CXCR4+/Flk-1+ sub-population is the 1^{st} bar and CXCR4−/Flk-1− sub-population is the 2^{nd} bar for each listed gene. (E-G): Spheroids from CXCR4⁻/Flk⁻ cells, morphologically consistent with less differentiated progeny, were quiescent (E, inset; n=35), in contrast to CXCR4⁺/Flk⁺ cells that formed expansive mesenchymal-like aggregation (E; n=17) and developed prominent beating areas (−40%) with sustained contractile activity (F). CXCR4⁺/Flk⁺ aggregates loaded with Fluo 4-AM demonstrated rhythmic intracellular calcium transients deconvoluted from line scans (G).

FIG. 8 is an illustration of an acute ischemic injury model produced by occlusion of the anterior coronary artery through an open thoracotomy in adult mice. The lower panels of FIG. 8 demonstrate the process of anterior coronary artery ligation and intra-venous infusion.

FIG. 9 depicts cardiac performance of three cohorts compared to pre-infarction condition. FIG. 9A is a bar graph of the results from the stress test. FIG. 9B contains echocardiograms at 4 weeks after infarction in animals treated with CXCR4⁺/Flk-1⁺ (+/+) or CXCR4⁻/Flk-1⁻ (−/−). FIG. 9C is a graph of the change in ejection fraction percent (ΔEF %) for the control (placebo infused animals), −/− and +/+treated animals.

FIG. 10 contains an echocardiogram (10A) and three graphs of cardiac function (10B-10D). FIG. 10B is a bar graph of the ejection fraction (EF, %), FIG. 10C is a bar graph of left ventricular diastolic dimensions (LVDd, mm), and FIG. 10D is a bar graph of heart to body weight ratio (Heart/BW, mg/g) comparing pre-MI levels with placebo infused and +/+ treated animals.

FIG. 11 is a FACS analysis of the negative control (top panel) and whole blood (bottom panel) using CXCR4 and Flk-1 antibodies.

### DETAILED DESCRIPTION

This document provides methods and materials related to cardiac cells and cells capable of differentiating into cardiac cells. For example, this document provides cells (e.g., CXCR4⁺/Flk-1⁺ stem cells) having the ability to differentiate into cardiac cells (e.g., cardiomyocytes), cardiac cells obtained from such cells, methods for making such cells, and methods for using such cells to provide heart tissue with cardiac cells. As described herein, selecting pre-programmed cardiac-specific progenitors from primitive stages of embryonic stem cell differentiation can provide an approach to harness an otherwise unrestricted repertoire. In particular, a selected pre-cardiac progenitor cell population can be identified according to a unique expression profile of cell surface biomarkers, CXCR4 and Flk-1. CXCR4 is a chemokine receptor to which SDF-1 binds. Flk-1, which also is known as KDR or VEGF-2 receptor, is an early mesoderm specific marker. These sorted progenitor cells differentiate into functional cardiac contractile tissue characterized by molecular expression of cardiac transcription factors, establishment of sarcomeres, and operational excitation-contraction coupling. These CXCR4⁺/Flk-1⁺ cardiac progenitors are distinct from CXCR4⁻/Flk-1⁻ counterparts that do not exhibit cardiac lineage specification. As such, the methods described herein can be used to identify a self-renewing transient pool of cardiopoietic progenitor cells, allowing a platform for targeted isolation of the primitive progeny that inherit an inductive cardiogenic program, prior to a recognizable cardiac phenotype. The identified dual biomarker can identify an early selected sub-population of cardiac progenitor cells from stage-specific in vitro embryonic stem cells prior to Myocardin, Mef2C, and Nkx2.5 gene expression.

This document also provides enriched populations of CXCR4⁺/Flk-1⁺ stem cells. As used herein, “enriched” with respect to a population of CXCR4⁺/Flk-1⁺ stem cells means that the population has at least a one fold increase (e.g., at least two, three, four, five, six, seven, eight, nine, ten, 15, 20, 25, 50, or 75 fold increase) in CXCR4⁺/Flk-1⁺ stem cells as compared to the crude population of stem cells from which the CXCR4⁺/Flk-1⁺ stem cells are isolated.

Cardiac cells can be any type of heart cells. For example, cardiac cells can be mammalian (e.g., human or murine) heart cells. In some cases, cardiac cells can be cardiomyocytes. Cells having the ability to differentiate into cardiac cells can be any type of cells having the ability to differentiate into cardiac cells. For example, cells having the ability to differentiate into cardiac cells can be mammalian (e.g., human) cells having the ability to differentiate into cardiac cells. In some cases, cells having the ability to differentiate into cardiac cells can be referred to as cardiopoietic cells. The term cardiopoietic cell used herein refers to a cell having the ability to differentiate into a cardiomyocyte.

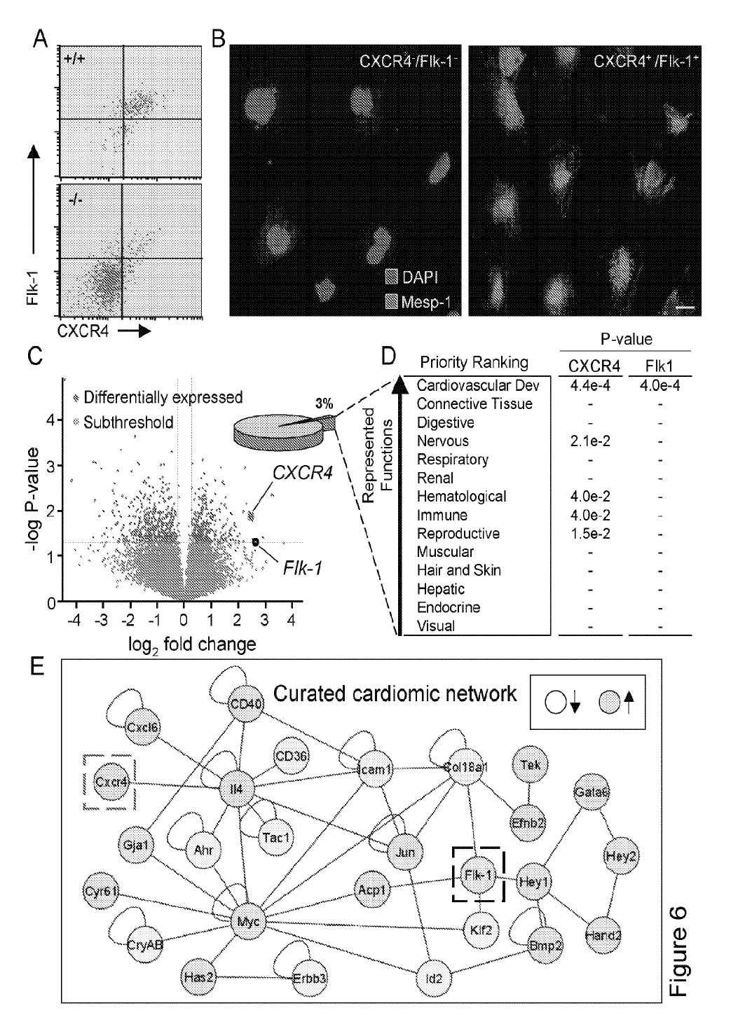

CXCR4⁺/Flk-1⁺ stem cells (e.g., CXCR4⁺/Flk-1⁺ embryonic stem cells) can have the ability to differentiate into cells that express, for example, Mef2C (myocyte enhancer factor 2C), GATA-4 (GATA binding protein 4), Myocardin, and Nkx2.5 (NK2 transcription factor related) polypeptides. In addition, as set forth in FIG. 6, one or more of the following genes can be up-regulated in CXCR4⁺/Flk-1⁺ stem cells undergoing differentiation relative to CXCR4⁻/Flk-1⁻ stem cells: Cxc16 (chemokine (C-X-C motif) ligand 6 (granulocyte chemotactic protein 2), CD40, IL4 (interleukin 4), Myc, Gja1 (gap junction protein, alpha 1), Cyr61 (cysteine rich, angiogenic inducer, protein 61), Has2 (hyaluronan synthase 2), Acp1 (acid phosphatase 1, soluble), Jun, Bmp2 (bone morphogenetic protein 2), Hand2 (heart and neural crest derivatives expressed transcript 2), Hey1 (hairy/enhancer-of-split related with YRPW motif 1), Hey2 (hairy/enhancer-of-split related with YRPW motif 2), Gata6 (GATA binding protein 6), Efnb2 (ephrin B2), and Tek (TEK tyrosine kinase, endothelial). As such, in some embodiments, CXCR4⁺/Flk-1⁺ stem cells can have the ability to differentiate into cells that express, for example, one or more of Cxc16, CD40, IL4, Myc, Gja1, Cyr61, Has2, Acp1, Jun, Bmp2, Hand2, Hey1, Hey2, Gata6, Efnb2, and Tek polypeptides at levels that are increased relative to that of CXCR4⁻/Flk-1⁻ stem cells.

In some embodiments, one or more of the following genes can be down-regulated in CXCR4⁺/Flk-1⁺ stem cells undergoing differentiation relative to CXCR4⁻/Flk-1⁻ stem cells: CryAB (crystallin, alpha B), CD36, Ahr (aryl-hydrocarbon receptor), Tact (tachykinin, precursor 1), Erbb3 (v-erb-b2 erythroblastic leukemia viral oncogene homolog 3), kam1 (intercellular adhesion molecule 1), Id2 (inhibitor of DNA binding 2, dominant negative helix-loop-helix protein), Klf2 (Kruppel-like factor 2), and Col18a1 (collagen, type XVIII, alpha 1). As such, in some embodiments, CXCR4⁺/Flk-1⁺ stem cells can have the ability to differentiate into cells that express, for example, one or more of CryAB, CD36, Ahr, Tac1, Erbb3, Icam1, Id2, Klf2, and Col18a1 polypeptides at levels that are decreased relative to that of CXCR4⁻/Flk-1⁻ stem cells.

Any appropriate method can be used to obtain CXCR4⁺/Flk-1⁺ stem cells. For example, CXCR4⁺/Flk-1⁺ stem cells can be derived from stem cells such as mammalian (e.g., human) stem cells using cell sorting techniques or cell panning techniques. CXCR4⁺/Flk-1⁺ stem cells can be obtained from any population of stem cells including, without limitation, embryonic stem cells, mesenchymal stem cells, and bone marrow stem cells. For example, stem cells can be incubated with anti-CXCR4 and anti-Flk-1 antibodies and a cell population positive for both CXCR4 and Flk-1 can be obtained using flow cytometry.

Once CXCR4⁺/Flk-1⁺ stem cells have been obtained, the cells can be monitored to determine whether or not they have the ability to differentiate into cardiac cells. For example, the CXCR4⁺/Flk-1⁺ embryonic stem cells can be assessed for the ability to differentiate into cells that express Mef2C, GATA-4, Myocardin, and Nkx2.5 polypeptides (or any combination thereof). Any appropriate method can be used to test cells for expression of a particular polypeptide including Western blotting, fluorescence-activated cell sorting (FACS), immunostaining, and laser confocal microscopy. CXCR4⁺/Flk-1⁺ cells also can be assessed for ability to differentiate into cardiac contractile tissue. For example, it can be determined if sarcomeres are established and/or operational excitation-contraction coupling develops. Operational excitation-contraction coupling is characterized by calcium transients and initiation of beating. See, Examples 1 and 6 of this document.

Any appropriate method can be used to provide cardiovascular tissue with cardiac cells, including transvascular routes and direct injections. For example, cardiac cells can be injected into the coronary artery, infused in the heart, administered systemically, or injected transendocardially. Cardiac cells can be provided to any cardiovascular tissue. For example, mammalian (e.g., human, monkey, horse, sheep, cow, pig, dog, cat, or rodent.) heart tissue can be provided with cardiac cells. In some embodiments, the cardiac cells are provided to injured heart tissue, e.g., heart tissue that has suffered from ischemic cardiomyopathy, myocardial infarction, or heart failure. In some embodiments, the injured tissue can be tissue damaged congenitally. In such an embodiment, the cardiac cells provided herein can be administered immediately after birth or prenatally. As described herein, intravenous infusion of CXCR4⁺/Flk-1⁺ cells can improve one or more of contractile function, myocardial structure, heart to body weight ratio, ejection function, and left ventricular diastolic dimensions. Without being bound to a particular mechanism, expression of CXCR4 and Flk-1 can target the CXCR4⁺/Flk-1⁺ cells to migrate into areas rich in expression receptor ligands (SDF-1 and VGEF), such as ischemic heart muscle.

Any type of cardiac cells can be administered to heart tissue. For example, autologous or heterologous cardiac cells can be administered to heart tissue. In some cases, CXCR4⁺/Flk-1⁺ stem cells (e.g., CXCR4⁺/Flk-1⁺ embryonic stem cells) or cells differentiated from CXCR4⁺/Flk-1⁺ stem cells can be administered to heart tissue.

In some embodiments, growth factors or cytokines can be administered to the heart tissue in combination with the CXCR4⁺/Flk-1⁺ stem cells. For example, growth factors or cytokines such as TNFα or γ-interferon, or any combination thereof can be administered to the heart tissue. The growth factors or cytokines can be administered to the heart tissue before or after cardiac cells are administered. In some cases, the growth factors or cytokines can be administered with the cardiac cells. While not being limited to any particular mode of action, growth factors and cytokines such as TNFα or γ-interferon can cause heart tissue to produce polypeptides that create an environment favorable for the differentiation into cardiomyocytes without producing tumor cells.

Methods described herein can include monitoring the recipient of the cells, for example, to determine if cardiac function is improving with treatment. Any method can be used to monitor the patient. For example, ejection fraction, heart to body weight ratio, or left ventricular diastolic dimensions can be assessed using, for example, echocardiography.

In some embodiments, changes in progenitor cell concentrations can provide diagnostic value. For example, CXCR4⁺/Flk-1⁺ stem cell levels can provide a predictor of the ability of an individual to recover from cardiac injury such as injury associated with myocardial infarction. In general, the number or percent of CXCR4⁺/Flk-1⁺ cells in a biological sample from a patient (e.g., a human patient) can be detected and compared to the number or percent of CXCR4⁺/Flk-1⁺ cells from a control population (e.g., the average number or percent of CXCR4⁺/Flk-1⁺ cells from a plurality of subjects without cardiac injury). Methods for detecting number or percent of CXCR4⁺/Flk-1⁺ cells are described herein. Suitable biological samples for measuring CXCR4⁺/Flk-1⁺ cells include, for example, blood (including whole blood, plasma, and serum). Thus, it can be determined if the number or percent of CXCR4⁺/Flk-1⁺ cells are increased, decreased, or the same as that of the control population. An increase in CXCR4⁺/Flk-1⁺ levels relative to that of the control population can be indicative of an improved ability to recover from the cardiac injury.

Cardiac cells described herein can be modified such that the cells produce one or more polypeptides or other therapeutic compounds of interest. As used herein, the term “polypeptide” refers to any chain of amino acids, regardless of length or post-translational modification. Typically, modified cells include an exogenous nucleic acid encoding the desired polypeptide (e.g., an angiogenic growth factor). For example, the exogenous nucleic acid can encode tumor necrosis factor α or γ-interferon. Therapeutic compounds include small molecules produced by polypeptides (e.g., prostaglandins or nitric oxide (NO)), as well as ribozymes and antisense nucleic acids. As a result, the modified cells can deliver any polypeptide or any therapeutic compound to the mammal for treating cardiac disease. In addition, marker polypeptides can be delivered to a patient to aid in diagnostic testing.

To modify the isolated cells such that a polypeptide or other therapeutic compound of interest is produced, the appropriate exogenous nucleic acid must be delivered to the cells. In some embodiments, the cells are transiently transfected, which indicates that the exogenous nucleic acid is episomal (i.e., not integrated into the chromosomal DNA). In other embodiments, the cells are stably transfected, i.e., the exogenous nucleic acid is integrated into the host cell's chromosomal DNA. The term “exogenous” as used herein with reference to a nucleic acid and a particular cell refers to any nucleic acid that does not originate from that particular cell as found in nature. In addition, the term “exogenous” includes a naturally-occurring nucleic acid. For example, a nucleic acid encoding a polypeptide that is isolated from a human cell is an exogenous nucleic acid with respect to a second human cell once that nucleic acid is introduced into the second human cell.

The exogenous nucleic acid can be transferred to the cells using recombinant viruses that can infect cells, or liposomes or other non-viral methods such as electroporation, microinjection, or calcium phosphate precipitation, that are capable of delivering nucleic acids to cells. In either case, the exogenous nucleic acid that is delivered typically is part of a vector in which a regulatory element such as a promoter is operably linked to the nucleic acid of interest. The promoter can be constitutive or inducible. Non-limiting examples of constitutive promoters include cytomegalovirus (CMV) promoter and the Rous sarcoma virus promoter. As used herein, “inducible” refers to both up-regulation and down regulation. An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent such as a protein, metabolite, growth regulator, phenolic compound, or a physiological stress imposed directly by, for example heat, or indirectly through the action of a pathogen or disease agent such as a virus. The inducer also can be an illumination agent such as light and light's various aspects, which include wavelength, intensity, fluorescence, direction, and duration.

Additional regulatory elements that may be useful in vectors, include, but are not limited to, polyadenylation sequences, translation control sequences (e.g., an internal ribosome entry segment, IRES), enhancers, or introns. Such elements may not be necessary, although they may increase expression by affecting transcription, stability of the mRNA, translational efficiency, or the like. Such elements can be included in a nucleic acid construct as desired to obtain optimal expression of the nucleic acids in the cell(s). Sufficient expression, however, may sometimes be obtained without such additional elements.

Vectors also can include other elements. For example, a vector can include a nucleic acid that encodes a signal peptide such that the encoded polypeptide is directed to a particular cellular location (e.g., the cell surface) or a nucleic acid that encodes a selectable marker. Non-limiting examples of selectable markers include puromycin, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture.

Viral vectors that can be used include adenovirus, adeno-associated virus (AAV), retroviruses, lentiviruses, vaccinia virus, measles viruses, herpes viruses, and bovine papilloma virus vectors. See, Kay et al. (1997) Proc. Natl. Acad. Sci. USA 94:12744-12746 for a review of viral and non-viral vectors. Viral vectors are modified so the native tropism and pathogenicity of the virus has been altered or removed. The genome of a virus also can be modified to increase its infectivity and to accommodate packaging of the nucleic acid encoding the polypeptide of interest.

Non-viral vectors can be delivered to cells via liposomes, which are artificial membrane vesicles. The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Transduction efficiency of liposomes can be increased by using dioleoylphosphatidylethanolamine during transduction. See, Felgner et al. (1994) J. Biol. Chem. 269:2550-2561. High efficiency liposomes are commercially available. See, for example, SuperFect® from Qiagen (Valencia, Calif.).

Cells described herein can be combined with packaging material and sold as a kit for providing cardiovascular tissue (e.g., human heart tissue such as injured human heart tissue) with CXCR4⁺/Flk-1⁺ stem cells or cardiomyocytes. Components and methods for producing articles of manufactures are well known. For example, a kit can include an enriched population of CXCR4⁺/Flk-1⁺ stem cells. Such a kit further can include sterile water or pharmaceutical carriers for administering such cells to mammalian heart tissue. Instructions describing how the enriched populations of CXCR4⁺/Flk-1⁺ stem cells are useful for treating injured heart tissue and improving cardiac function can be included in such kits.

In other embodiments, a kit includes reagents for isolating enriched populations of CXCR4⁺/Flk-1⁺ stem cells. For example, a kit can include antibodies that bind to a CXCR4 polypeptide (e.g., human or mouse CXCR4) and/or antibodies that bind to a Flk-1 polypeptide (e.g., human or mouse Flk-1). In addition, the articles of manufacture may further include reagents such as secondary antibodies, buffers, indicator molecules, and/or other useful reagents for isolating enriched populations of CXCR4⁺/Flk-1⁺ stem cells. Instructions describing how the various agents are effective for isolating enriched populations of CXCR4⁺/Flk-1⁺ stem cells also may be included in such kits.

The invention will be further described in the following examples, which do not limit the scope of the invention described in the claims.

### EXAMPLES

### Example 1

### Methods and Materials

Embryonic Stem Cell Derived Cardiopoiesis

Murine embryonic stem cells (ESC) (GCR8 and R1-derived lines) were maintained in Glasgow's Minimum Essential Medium (BioWhittaker-Cambrex, Walkersville, Md.) supplemented with pyruvate and L-glutamine (Cellgro, Mediatech, Inc. Herndon, Va.), non-essential amino acids (Cellgro, Mediatech, Inc. Herndon, Va.), 13-mercaptoethanol (Sigma-Aldrich, St Louis, Mo.), 7.5%-10% fetal calf serum (FCS, Invitrogen Corporation, Carlsbad, Calif.) and leukemia inhibitory factor (LIF; ESGRO, Chemicon International, Inc, Temecula, Calif.). ESC were subsequently differentiated into three-layer embryoid bodies using the hanging-drop method in differentiation media supplemented with 20% FCS and TNF-α (Sigma-Aldrich, St Louis, Mo.) as described elsewhere (Behfar et al., (2005) Ann. N.Y. Acad. Sci., 1049, 189-198). A time course of embryonic stem cell differentiation starting with undifferentiated embryonic stem cells maintained in LIF was obtained from cell aggregates in suspension at days 3-5 and from plated embryoid bodies at days 7-9. Dual interface Percoll gradient was used to enrich sarcomere-rich high density cardiomyocytes (Behfar et al., (2002) FASEB J., 16, 1558-1566 and Perez-Terzic et al., (2003) Circ. Res., 92, 444-452) from the lower density sarcomere-poor cardiopoietic phenotype from day 7 embryoid bodies. ESC-derived progeny were fixed in 3% paraformaldehyde, permeabilized with 1% Triton X-100, and immunostained with antibodies specific for cardiac transcription factors Mef2c (1:400, Cell Signaling Technologies, Danvers, Mass.), Nkx 2.5 (1:150, Santa Cruz Biotechnology Inc., Santa Cruz, Calif.), Mesp-1 (1:500, Novusbio, Littleton, Colo.), AFP (1:100, Cell Signaling Technologies, Danvers, Mass.), and sarcomeric protein α-actinin (1:1,000, Sigma-Aldrich, St Louis, Mo.) along with DAPI staining to visualize individual nuclei (Behfar et al., (2005) Ann. N.Y. Acad. Sci., 1049, 189-198). Microscopy was preformed using an LSM 510 laser scanning confocal microscope (Carl Zeiss Inc., Thornwood, N.Y.).

Genomics

Total RNA was extracted from cell populations at selected developmental stages using a combination of gDNA Eliminator and RNeasy columns (Qiagen, Valencia, Calif.). cDNA was prepared from total RNA samples using MMLV Reverse Transcriptase (Invitrogen Corporation, Carlsbad, Calif.). Samples were subjected to microarray analysis by labeled cRNA hybridization to the mouse genome 430 2.0 GeneChip (Affymetrix, Inc, Santa Clara, Calif.). Embryonic stem cells in the presence and absence of LIF, cardiopoietic cells, and cardiomyocytes were analyzed using cDNA from triplicate biological samples. Real-time PCR was performed using a standard TaqMan° PCR kit protocol on an Applied Biosystems 7900HT Sequence Detection System (Applied Biosystems, Foster City, Calif.). The 50 μL PCR included 3 μL RT product, 25 μL×TaqMan° Universal PCR Master Mix (Applied Biosystems, Foster City, Calif.), 19.5 μL RNase free water and 2.5 μL TaqMan° Gene Expression Assays (pre-designed, pre-optimized probe and primer sets for each gene of interest, Applied Biosystems, Foster City, Calif.). TaqMan® Gene Expression Assays contained 2 unlabeled PCR primers (900 nM each final concentration) and 1 FAM dye-labeled TaqMan® MGB probe (250 nM final concentration). The reactions were incubated in a 96-well plate at 95° C. for 10 minutes, followed by 40 cycles of 95° C. for 15 seconds and 60° C. for 1 minute. All reactions were run in triplicate. The threshold cycle (CT) was defined as the fractional cycle number at which fluorescence passes the fixed threshold. TaqMan® CT values were converted into relative fold changes determined using the 2^{-ΔΔC}_{T} method, normalized to mouse GAPDH (P/N 4352662-0506003). Prototypical genes representative of each germinal layer were selected for analysis, which included Lhx1 (Mm00521776_m1), Nkx2.5 (Mm00657783_m1), Gata4 (Mm00484689_m1), Myocd (Mm00455051_m1), Oct4 (Mm00658129_gH), Fgf4 (Mm0000438917_m1), Cxcr4 (Mm01292123_m1), Mef2c (Mm01340839_m1), Gsc (Mm00650681_g1), Sox17 (Mm00488363_m1), Sox7 (Mm00776876_m1), Flk1 (Mm00440099_m1), Sox1 (Mm00486299_s1), Sox2 (Mm00488369_s1), Tbx5 (Mm00803521_m1), Myh11 (Mm00443013_m1), and PECAM (Mm00476702_m1). Comparisons between groups was performed by Student's t tests with 95% confidence intervals. DNA gels for Cxcr4 were run after samples were normalized to GAPDH.

Bioinformatics and Network Analysis

Gene expression changes of microarray data acquired using the GeneChip Scanner 3000 (Affymetrix, Inc, Santa Clara, Calif.) were profiled with the Genespring GX 7.3 analysis software suite (Agilent Technologies). The derived gene list was limited to report transcripts with expression levels above background and then subjected to 1-way ANOVA, using a Benjamini-Hochberg post hoc multiple testing correction for all P<0.01. Filtered significant genes were delimited by flag value, excluding those absent (A) in all samples for all stages. Gene Ontology Consortium designated membrane-associated transcripts (“integral to membrane”) were reproducibly present (P) in all three samples at the cardiopoietic stage. Co-ordinated gene profiles hierarchically assembled into an expression heatmap using a K-means clustering algorithm. Genes were selected from membrane-restricted genes according to normalized expression values higher than 1.5-fold. Significantly enriched Kyoto Encyclopedia of Genes and Genomes (KEGG) pathways within the derived upregulated cluster (cytokine, adhesion, Jak/STAT) were identified with associated P-values as a measure of similarity between the KEGG-established pathway and the experimentally derived list, with (P) calculated as follows:

$$\frac{1}{( \begin{matrix}u \\ n\end{matrix} )} {\sum }_{i = k}^{n} ( \begin{matrix}m \\ i\end{matrix} ) ( \begin{matrix}u - m \\ n - i\end{matrix} )$$

The probability (P) of overlap corresponds to (k) or more genes between (n) and (m) gene lists when randomly sampled from a universal list (u). Lower P-values indicate that gene overlap is more significant. Comparison among membrane restricted, cytokine clustered and quality filtered cardiopoietic lists identified overlapping transcripts, 69% of which exhibited increased expression during cardiopoiesis. Using Ingenuity Pathways Analysis, an established network analysis program, molecular interactions of membrane-associated, upregulated cytokine clustered genes were examined in the cardiopoietic stage. These relationships were recompiled into a format usable by the Institute for Systems Biology Cytoscape 2.2 software (http://www.cytoscape.org) and expressed as a localized network. Murine phenotypes associated with the 11 identified transcripts were investigated through bioinformatic mining of the Mouse Genome Informatics database (www.informatics.jax.org) to identify impacts of transgenic candidate gene knockouts on cardiac differentiation for ultimate restriction of the transcript pool.

Flow Sorting Embryonic Stem Cells

Embryonic stem cells were placed in 25 μL hanging drops (2,500 drops per experiment) that initially contained 250 cells each and were cultured for 2 days in 20% FCS (Invitrogen Corporation, Carlsbad, Calif.) supplemented with 30 ng/ml TNF-α (Invitrogen Corporation, Carlsbad, Calif.) in the absence of LIF, then allowed to differentiate further as large aggregates in suspension for 3 additional days. Aggregates were washed in PBS and then dissociated using non-enzymatic dissociation buffer (Invitrogen Corporation, Carlsbad, Calif.) for 10 minutes at 37° C. Aggregates were triturated using 10 mL pipette until a single cell suspension was obtained. Embryonic stem cell-derived cells were spun down at 1000×g for 5 minutes and resuspended in propagation media (7.5% FCS) for 10 minutes to allow cells to recover. Cells were divided into aliquots for negative control (secondary only) and single positive controls (Flk-1 and CXCR4) each containing 300,000 cells. The remaining cells (2×10⁷) obtained from initial aggregates were collected and immuno-stained for both Flk-1 and CXCR4 expression. Cells were washed with PBS and resuspended in 1 mL PBS which contained goat-CXCR4 antibody (1:150, Abcam, Cambridge, Mass.), placed on ice for a 30-minute incubation, followed by single wash with 10 mL PBS. Secondary anti-goat Alexa 488 (1:500, Molecular Probes, Invitrogen Corporation, Carlsbad, Calif.) and PE-conjugated primary antibody for Flk-1 (1:200, BD Biosciences, San Jose, Calif.) was incubated on ice for 30 minutes, followed by single 10 mL PBS wash. Cells were isolated using a FACS Vantage SE flow cytometer (BD Biosciences, San Jose, Calif.). Alexa-488 was excited with a 488 nm argon laser and detected through a 530/30 nm bandpass filter. PE was excited with the 488 nm laser line and detected through a 575/26 bandpass filter. Forward and side scatter parameters were used to gate on viable cell population sorted into subpopulations. Once collected in standard culture media, cells were centrifuged and frozen in liquid nitrogen for RNA isolation and mircroarray analysis. Alternatively, fresh cells were suspended in propagation media, diluted to 400,000 cells/ml, and 25 μl drops were suspended for 48 h prior to plating the re-aggregates on 0.1% gelatinized plates with or without visceral endoderm-like cells. Derived from an F9 cell population (ATCC) with retinoic acid (1 μM), dbcAMP (0.5 mM) and theophylline (0.5 mM), with phenotype confirmed through comparison with END-2 cells (Behfar et al., J. Exp. Med. 2007 204:405-420); Mummery et al. Circulation 2003 107:2733-2740), these cells were used to recapitulate the embryonic environment for 4 days of in vitro differentiation to complete the 9-day protocol.

Ontological and Network Analysis

Differentially expressed genes (P<0.05) were excluded from subthreshold transcripts using Volcano plot analysis, according to a minimum 1.5-fold change, and ontologically dissected to determine physiological system priority emphasized within changing transcripts. Significant association of sortable cell surface biomarkers was determined, through Ingenuity Pathways Analysis, within each prioritized physiological system. Molecular interactions of expression profiles comprising Cardiovascular Development were examined and formatted for Cytoscape 2.2, which provided an ad hoc network map of integrated up- and down-regulated pro-cardiac candidate genes.

Beating Activity and Calcium Transients

Sorted subpopulations of stem cell-derived progenitor aggregates co-cultured with visceral endoderm-like cells were monitored daily. Beating activity was recorded at 20 frames per second with phase contrast microscopy using a Zeiss Observer.Z1 microscope with ApoTome and live cell imaging system controlled at 37° C. Once beating activity was recorded, cells were loaded with the Ca²⁺-selective probe fluo-4-acetoxymethyl ester (Molecular Probes) with a final concentration of 5 μM in serum-free cell culture media and incubated for 30 min at 37° C. before washing in PBS and allowed to recover in standard culture media for 1 h. Using the temperature controlled live cell imaging system on a Zeiss ApoTome microscope, fluo4-loaded cells were illuminated with a mercury lamp at 400±20 nm and fluorescence was recorded with a AxioCam MRm camera and AxioVision software. Intracellular Ca²⁺ transients were deconvoluted as a function of time and analyzed with MetaMorph® software (Universal Imaging Corporation, West Chester, Pa.).

### Example 2

### Systems Bioinformatics Prioritizes a Chemokine Cluster in Embryonic Stem Cell Cardiopoiesis

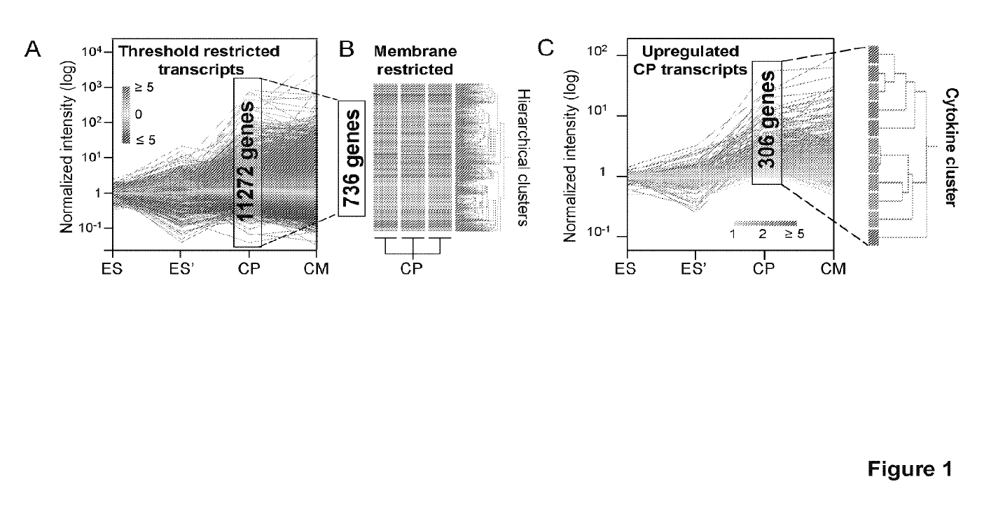

Blastocyst-derived embryonic stem cells maintain their pluripotent capacity in vitro when treated with leukemia inhibitory factor, and upon removal of this mitogen, differentiate into embryoid bodies recapitulating the three germinal layers obtained with embryonic development (Smith et al., (1988) Nature, 336, 688-690). Within the heterogeneous lineages of a differentiating embryoid body, cardiogenic priming with TNF-α expedites cardiac commitment of embryonic stem cells. These progenitor cells, which can be referred to as cardiopoietic stem cells, engage the early cardiogenic program following nuclear translocation of cardiac transcription factors, and are identified as an intermediate stage in cardiac differentiation preceding sarcomerogenesis of the mature cardiomyocytes. Compared to their parental embryonic stem cells in the presence or absence of leukemia inhibitory factor, genome-wide screening revealed that cardiopoietic progenitors display a distinctive pre-cardiac transcriptome profile that gradually acquires the genomic fingerprint of cardiomyocytes (FIG. 1A). The inclusive pool of transcripts present at the cardiopoietic stem cell stage was represented by a spectrum of 11,272 genes mapped with microarray analysis (FIG. 1A). Restricting the cardiopoietic profile to individual transcripts encoding polypeptides expressed within the cellular membrane resolved 736 genes hierarchically clustered as potential surface candidate markers (FIG. 1B). Within this candidate marker pool, dynamic mapping demonstrated 306 genes with a significantly increased gene expression profile at the cardiopoietic stage (FIG. 1C). By mining experimental systems expression profiles with bioinformatically curated functional families, gene clusters containing cardiopoiesis-upregulated plasmalemmal signaling components were unmasked (FIG. 1C). Specifically, this screening approach identified three overrepresented functional clusters at the cardiopoietic stage, i.e., the cytokine/chemokine receptor signaling (p=8.65×10⁻⁵) ahead of cell adhesion (p=6.46×10⁻³) and JAK/STAT (p=2.91×10⁻³) clusters. Thus, in silico analysis of the transcriptome in cardiac differentiation of embryonic stem cells prioritized the prominence of the cytokine/chemokine cluster during cardiopoiesis.

### Example 3

### Deconvoluted Chemokine Cluster Reveals Cardiogenic Linkage with CXCR4 Receptor

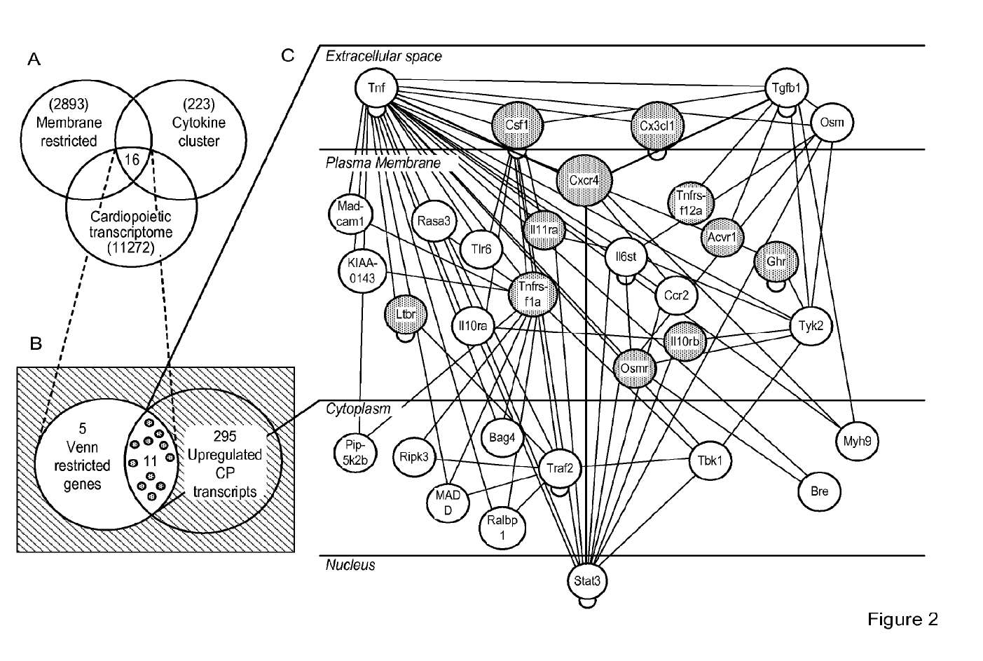

The cytokine/chemokine cluster incorporated 4% of the total 306 upregulated membrane-restricted genes identified at the cardiopoietic stage of differentiation (FIG. 1). An unbiased comparison among the Kyoto Encyclopedia of Genes and Genomes (KEGG) pathway designated cytokine/chemokine family (223 genes), the Gene Ontology (GO) Consortium roster of genes integral to membrane (2,893 genes), and the experimentally determined transcripts in cardiopoietic stem cells (11,272 genes) independently verified gene candidates within the cytokine family (FIG. 2A). Venn diagram resolution of these intersecting gene cohorts demarcated 16 overlapping transcripts, verifying the 11 genes of the identified cytokine cluster at the cardiopoietic stage of differentiation i.e., CXCR4, Il1 lra1, Ghr, Ltbr, Tnfrsf1a, CX3cl1, Acvr1, Tnfrsf12a, Il1 Orb, Osmr, and Csf1, with the remaining 5 neither upregulated nor present (FIG. 2B). Linkage of the 11 identified cytokine clustered genes to cardiovascular development was interrogated by mining the annotated murine phenotypes within the Mouse Genome Informatics database (www.informatics.jax.org). While 10 genes were associated with non-specific systemic disruptions in organismal growth and cell size (Ghr, Tnfrsf1a, Tnfrsf12a, Acvr1), abnormalities in immunological and hematopoietic systems (Ltbr, Csf1, Osmr, MOO), vascular defects in fetalplacental development (IL11ra1), and atherosclerotic susceptibility (CX3cl1), cardiac-specific abnormalities with impaired cardiogenesis were identified in the CXCR4-null mutant (Zou et al., (1998) Nature, 393, 595-599). The chemokine receptor, CXCR4, integrated into a cytokine/chemokine cluster generating a non-stochastic, pro-cardiogenic network anchored by the extracellular TGF-β/TNF-α signaling cascades and nuclear JAK/STAT molecules (FIG. 2C). Thus, bioinformatic deconvolution of the cardiopoietic gene pool identified CXCR4 as a uniquely qualified biomarker induced during cardiac transformation and integrated into a cardiogenic signaling network.

### Example 4

### Cardiopoietic Induction of CXCR4 is Unique to the Chemokine Receptor Family and Precedes Canonical Cardiac Marker Expression

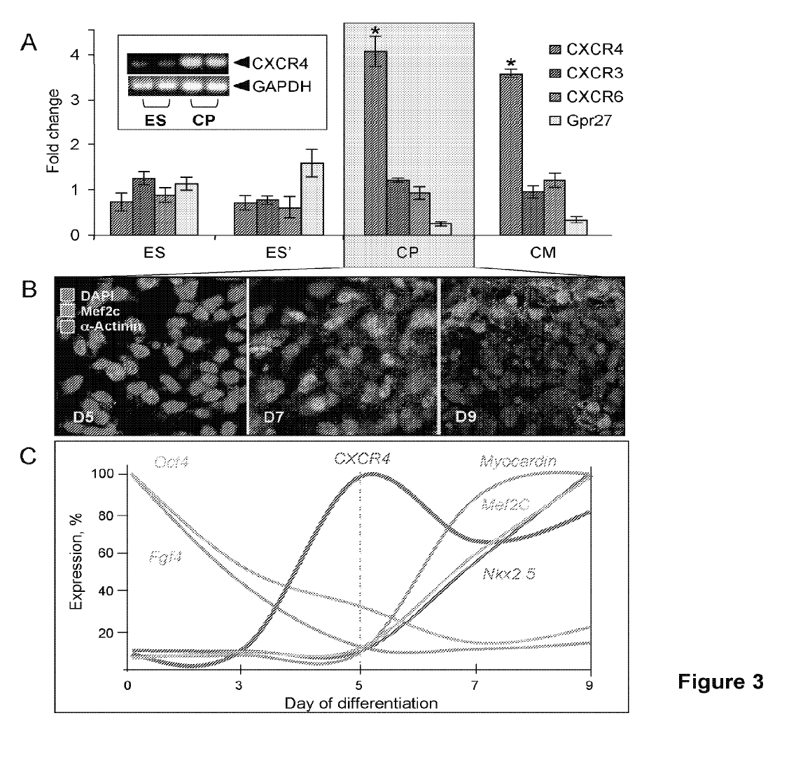

To determine whether induction of CXCR4 during cardiogenesis is unique in the chemokine family, gene microarrays labeled with chemokine receptor probes quantified individual expression levels during embryonic stem cell cardiac differentiation. Transcripts of only four chemokine receptor genes, i.e., CXCR4, CXCR3, CXCR6, Gpr27, were detected in early cardiopoiesis (FIG. 3A). While three had static expression profiles (CXCR3, CXCR6, Gpr27) between stages, CXCR4 uniquely displayed a dynamic expression profile reaching a peak at the cardiopoietic stage with sustained expression during cardiac maturation (FIG. 3A). RT-PCR verified the significant induction of CXCR4 mRNA expression as embryonic stem cells transform from the pluripotent to the cardiopoietic phenotype (FIG. 3A inset). To trace the temporal expression profile of the CXCR4 gene during cardiopoiesis, mRNA expression was quantified using quantitative RT-PCR at sequential stages of embryonic stem cell differentiation (FIG. 3B-C). At day 0, pluripotent genes (Oct-4, Fgf-4) demonstrated maximal transcript levels that progressively decreased with differentiation (FIG. 3C). Loss of pluripotent markers at day 5 was associated with a sudden surge in CXCR4 gene expression (FIG. 3C). CXCR4 expression was coupled, by day 7, with induction of cardiac-specific transcripts (Mef2c, Myocardin, and Nkx2.5; FIG. 3C). Initiation of cardiogenic transformation was verified by nuclear translocation of the canonical cardiac transcription factor Mef2c, detected by immunostaining on confocal microscopy (FIG. 3B). Expression of sarcomeric a-actinin at day 9 correlated with the onset of spontaneous beating at day 8, indicating ongoing maturation of the cardiac phenotype. Thus, an early stage of embryonic stem cell differentiation selectively induces the CXCR4 chemokine receptor prior to the genetic switch into a cardiogenic program.

### Example 5

### Pre-Cardiac CXCR4+ Progeny Co-Express Mesoderm Marker, Flk-1

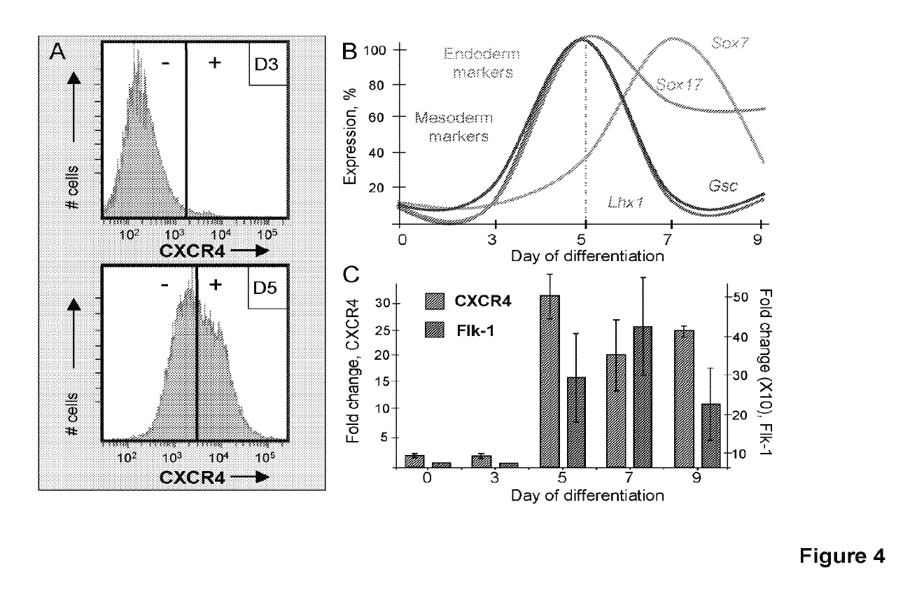

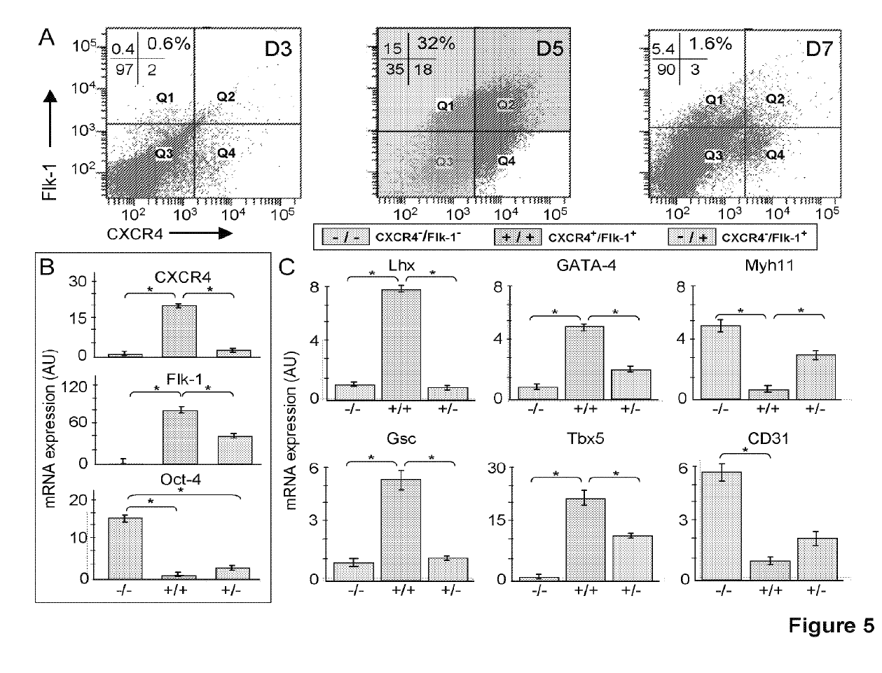

Fluorescence activated cell sorting quantified CXCR4 polypeptide expression in embryonic stem cells at different stages of differentiation (FIG. 4). As predicted by transcriptome analysis (FIG. 3), CXCR4 polypeptide expression was not significantly detected before day 5 of embryoid body differentiation. Less than 5% of cells from three-day-old embryoid bodies were positive for CXCR4 polypeptide; however, by day 5, 50% of total embryoid body-derived cells expressed abundant levels of cell surface CXCR4 polypeptide (FIG. 4A). This notable induction indicated a phenotypic transition within the embryoid body from a CXCR4-negative, primordial stem cell population to a mixture of CXCR4-positive differentiating progenitors. Indeed, at day 5 of embryonic stem cell differentiation, a marked induction of genes indicative of mesoderm (Goosecoid, Lhx1) as well as endoderm (Sox 7, Sox17) was observed (FIG. 4B), demonstrating a major branch point for germ layer specification that recapitulated the gene expression profile of a gastrulating embryo. The early mesoderm specific marker Flk-1, also known as KDR or VEGF-2 receptor (Yamaguchi et al., (1993) Development, 118, 489-498), demonstrated an mRNA transcription profile paralleling CXCR4 (FIG. 4C). Flk-1 was applied as a probe for the mesoderm-specific cells within the CXCR4-positive mixture that includes both mesoderm and endoderm progeny (McGrath et al., (1999) Dev. Biol., 213, 442-456.). Specifically, FACS analysis detected membrane-associated protein expression of Flk-1, increasing between days 3 and 5 (FIG. 5) congruent with the induction of CXCR4 polypeptide expression. In particular, the subpopulation that co-expressed both CXCR4 and Flk-1 was <1% at day 3 of differentiation, peaked at ±30% by day 5, and decreased by day 7 (FIG. 5A). The embryonic stem cell derived population that co-expressed both CXCR4 and Flk-1 peaked at 30% by day 5 (FIG. 5), two days prior to expression of cardiac-specific genes. Thus, the phenotypic switch of pluripotent embryonic stem cells to a CXCR4-positive heterogenous mixture signifies a transformation to lineage-determining programs in which Flk-1 transient co-expression flags a pool of mesoderm cells equipped with the cardiogenic chemokine receptor.

### Example 6

### CXCR4+/Flk-1+ Progeny Inherits a Cardiopoietic Program Securing Cardiogenesis

Differentiating native embryoid bodies were dissociated at day 5, and sorted according to CXCR4 and Flk-1 expression to isolate pools of double-positive CXCR4⁻/Flk-1⁻, double-negative CXCR4⁻/Flk-1⁻, and CXCR4⁻/Flk-1⁺ sub-populations (FIG. 6A) for gene expression profiling. As quality control to ensure effective sorting, CXCR4⁻/Flk-1⁻ cells demonstrated quantitative gene expression differences in CXCR4 and Flk-1 compared to the CXCR4⁻/Flk-1⁻ subpopulation; CXCR4⁻/Flk-1⁺ cells expressed higher levels of Flk-1 compared to CXCR4⁻/Flk-1⁻ counterparts (FIG. 5B). The pluripotent marker Oct-4, while abundant in CXCR4⁻/Flk-1⁻ cells, was significantly downregulated in both CXCR4⁻/Flk-1⁻ and CXCR4⁻/Flk-1⁺ subpopulations (FIG. 5B). Moreover, the mesoderm-specific markers Lhx and Gsc were expressed at significantly elevated levels in CXCR4⁻/Flk-1⁻ compared to CXCR4⁻/Flk-1⁻ cells (FIG. 5C). Furthermore, transcription factors GATA-4 and Tbx5, associated with the embryonic primary heart field, were significantly enriched in CXCR4⁺/Flk-1⁺ cells when compared to either CXCR4⁻/Flk-1⁻ or CXCR4⁻/Flk-1⁺ subpopulations, indicating that the pre-cardiac mesoderm genotype was segregated with cells expressing the combination of CXCR4 and Flk-1 (FIG. 5C). The cardiogenic profile in CXCR4⁺/Flk-1⁺ cells was separated from the phenotypic expression of vascular smooth muscle and endothelium markers, demonstrated by decreased expression of Myh11 and CD31 (PECAM) genes compared to the CXCR4⁻/Flk-1⁺ subpopulation (FIG. 5C). Thus, co-expression of CXCR4 and Flk-1 tags a mesoderm-restricted subpopulation of progenitors during differentiation of embryonic stem cells that demonstrates a unique enrichment of genes associated with early embryonic heart development.

A quality-controlled progenitor pool (FIG. 6A) was used to further document characteristic features of a developing cardiac phenotype supported by robust expression of the pre-cardiac mesoderm marker Mesp-1 in CXCR4⁻/Flk-1⁻ compared to CXCR4⁻/Flk-1⁻ subpopulations (FIG. 6B). Global transcriptome comparison of the two subpopulations sorted at day 5 of differentiation (FIG. 6A) demonstrated an overall 97% similarity with significant changes in a set of 818 genes (FIG. 6C and inset). This differentially expressed gene set represents 14 distinct physiological systems identified by gene ontology analysis (FIG. 6D). Priority ranking, calculated according to the number of genes contributing to functional clusters established predominance of “Cardiovascular Development” as the most significant represented functional system (FIG. 6D). Significant co-expression of both CXCR4 and Flk-1 was exclusive to the “Cardiovascular Development” classification, as interrogation of all other systems involved either CXCR4 alone or neither of the markers (FIG. 6D). Curated analysis of transcripts specifying “Cardiovascular Development” generated a network integrating CXCR4 and Flk-1 along with 16 other upregulated and 9 downregulated genes (FIG. 6E). Cross-referencing with the Mouse Genome Informatics database (www at informatic.jax.org) revealed that the majority of network nodes (14/27) were directly associated with cardiovascular development (FIG. 6E). Thus, the CXCR4⁻/Flk-1⁻ subpopulation harbors a defining transcriptome fingerprint characterized by overrepresentation of cardiovascular developmental genes.

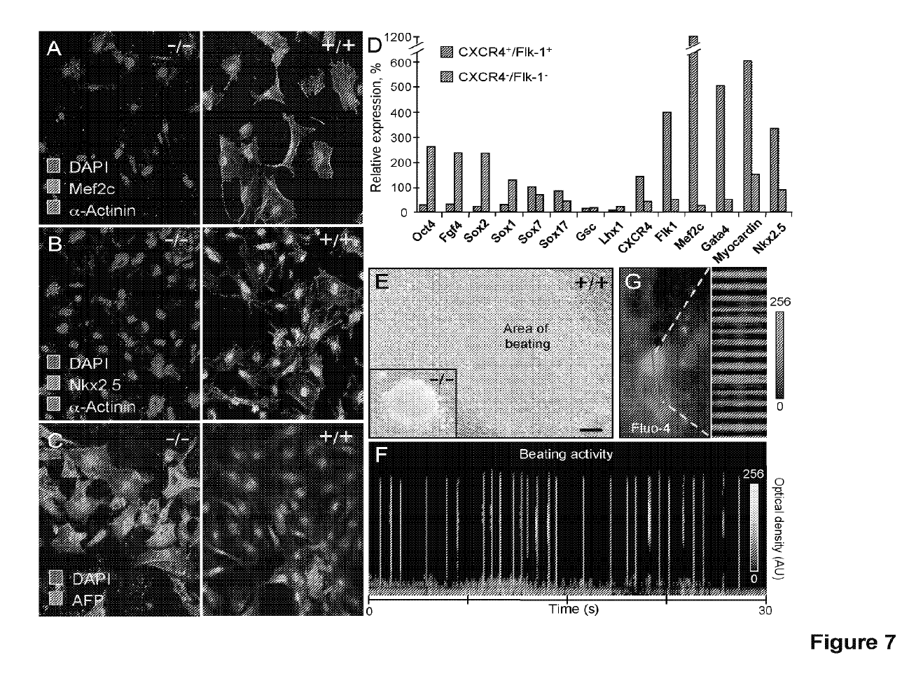

The physiological significance of the primitive cardiac developmental network intrinsic to the CXCR4⁻/Flk-1⁻ progeny was examined through comparative differentiation with CXCR4⁻/Flk-1⁻ cells. Allowed to complete differentiation in monolayers following cell sorting, CXCR4⁻/Flk-1⁻ progeny formed mesenchymal-like cellular structures containing cytoplasmic cardiac a-actinin and nuclear localized cardiac transcription factors, Mef2c and Nkx2.5 (FIG. 7A and FIG. 7B). In contrast, CXCR4⁻/Flk-1⁻ counterparts were devoid of the cardiogenic phenotype (FIG. 7A and FIG. 7B), and demonstrated alternative lineage-specification characterized by differential expression of the endoderm-specific marker AFP absent from CXCR4⁻/Flk-1⁻ progeny (FIG. 7C). Confirming distinct cardiac-lineage specification, RT-PCR data validated the disparate genetic programs of the CXCR4⁻/Flk-1⁻ progeny after 9 days of differentiation with robust expression of vital genes encoding for cardiac transcription factors (Mef2C, GATA-4, Myocardin and Nkx2.5), without significant presence of ectodermal/neuroectoderm (Sox1 and Sox2) and endodermal (Sox7 and Sox17) lineage-specific markers (FIG. 7D). In contrast, the CXCR4⁻/Flk-1⁻ progeny, while depleted of cardiac markers, displayed high levels of pluripotent genes (Oct 4, Fgf4) typical of the primordial germ layer and genes (Sox1, Sox2) of the neuroectoderm lineage (FIG. 7D).

To monitor cardiac maturation and acquisition of functional cardiogenic traits, the mesoderm-derived subpopulation of CXCR4⁻/Flk-1⁻ sorted progenitors, now devoid of endoderm components, were co-cultured on visceral endoderm-like cells to recapitulate embryonic endoderm environment (FIG. 7E). CXCR4⁻/Flk-1⁻ progeny differentiated as layers of aggregated tissue containing beating areas, in contrast to CXCR4⁻/Flk-1⁻ progeny limited to quiescent sphere-like structures that persisted beyond an extended 21-day differentiation (FIG. 7E inset). Beating activity (FIG. 7F) was recorded in ˜40% of CXCR4⁻/Flk-1⁻ clusters. Calcium transients, absent from CXCR4⁻/Flk-1⁻ progeny, were detected in CXCR4⁻/Flk-1⁻ aggregates with initiation of beating (FIG. 7G), indicating operational excitation-contraction coupling. Thus, dual expression of CXCR4/Flk-1 biomarkers separate pluripotent, neuroectoderm and/or endoderm phenotypes from a cardiopoietic lineage. Together, these results indicate that the CXCR4+/Flk-1+ sub-population, sorted from embryoid bodies at day 5 of differentiation, inherit a cardiopoietic program driving definitive cardiac transformation within embryonic stem cells.

Individual markers are however commonly associated with multiple lineages during stem cell differentiation, and singularly may not be sufficient to distinguish lineage specification. In this regard, the overlapping expression kinetics of CXCR4 and Flk-1 provided a highly specific dual biomarker approach that identified a pro-cardiac population. Spatiotemporal expression patterns of CXCR4 and Flk-1 were consistent with pre-cardiac mesoderm in the developing embryo in which Flk-1 expression is limited to mesoderm derivatives and excludes CXCR4-expressing endodermal progenitors. Simultaneous co-expression for CXCR4/Flk-1 preceded expression of conventional cardiac genes, including Myocardin, Mef2C and Nkx2.5, identifying this biomarker pair as an early predictor of cardiac fate. Added cardiac specificity is demonstrated by segregating the Flk-1 population according to CXCR4 expression to obtain further enriched cardiopoietic progenitors containing genes, such as Mesp-1, GATA-4 and Tbx5, associated with pre-cardiac mesoderm and the primary heart field.

Indeed, isolated pre-cardiac mesoderm cells, according to CXCR4/Flk-1 co-expression, revealed distinctive genetic features of a significant cardiac developmental potential, absent in the CXCR4⁻/Flk-1⁻ counterpart population. Upregulated pro-cardiac genes that include BMP-2, Myc, Hand2, and GATA-6 integrated into an early cardiac network, supported by downregulation of molecules that modulate differentiation (Id2), antagonize TGF-13 signaling (KLF2), or participate in oncogenesis (ERBB3). In this way, a primitive network, exposed by tandem CXCR4/Flk-1 dependent-selection, demonstrated a significant emerging property of pre-cardiac lineage specification with concomitant pluripotential deactivation. The physiological significance of cardiopoietic network engagement by the CXCR4⁻/Flk-1⁻ subpopulation was validated through successful cardiac phenotype maturation with development of associated beating activity and calcium transients, hallmarks of proper cardiac differentiation, in contrast to CXCR4⁻/Flk-1⁻ cells that were deprived of cardiogenic capacity. Thus, CXCR4⁻/Flk-1⁻ biomarkers identify progenitor cells programmed by pro-cardiogenic genes from the pluripotent state.

In summary, selecting a purified progenitor cell population predicted to become cardiac committed has therapeutic benefits over erratic multi-lineage stem cells in cardiac repair. Embryonic stem cells provide a unique opportunity to dissect progenitor cells from in vitro model systems with sufficient quantities of cells to probe primordial decisions of cellular fate that are critical for therapeutic translation. The results provided herein couple the innate genetic characteristics of the pre-programmed mesoderm identified by Flk-1 expression with primordial migratory instincts encoded by CXCR4 expression to identify a uniquely equipped pool of transient cardiac progenitors.

Establishing this unique tandem biomarker approach provides a platform to generate stage-specific tissue-predetermined progenitors from parental stem cells for cardiac regeneration.

### Example 7

### Intra-Venous Infusion of CXCR4⁻/Flk-1⁻ Cardiac Progenitors Repairs Myocardial Infarct

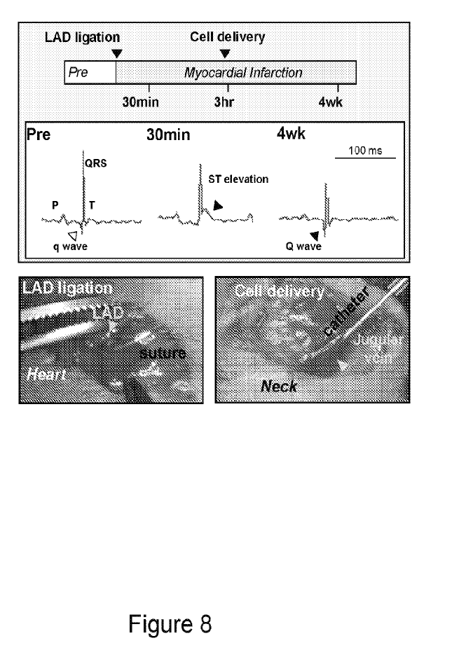

To test the in vivo therapeutic potential of CXCR4⁻/Flk-1⁻ cardiac progenitors, a mouse model of ischemic cardiac injury was developed. As illustrated in FIG. 8, this model consists of acute ischemic injury produced by occlusion of the anterior coronary artery through an open thoracotomy in adult mice. Mice underwent echocardiography and electrophysiology to determine post-infarction cardiac function after 30 minutes of occlusion. After 3 hours, mice were randomized to three arms: 1) CXCR4⁻/Flk-1⁻ progenitors, 2) CXCR4⁻/Flk-1⁻ progenitors, or 3) placebo injected. In blinded study design, infusion therapy was performed through intra-venous route via direct access through the internal jugular vein in the neck with injection solution containing no cells or cells at concentrations of 200,000 to 1,000,000 per 300 μl. FIG. 8 illustrates a schematic of this protocol and demonstrates the process of anterior coronary artery ligation and intra-venous infusion. This model recapitulates the clinically relevant acute myocardial infarction with ST-segment elevation within 30 minutes of occlusion, and Q-wave formation by 4-weeks.

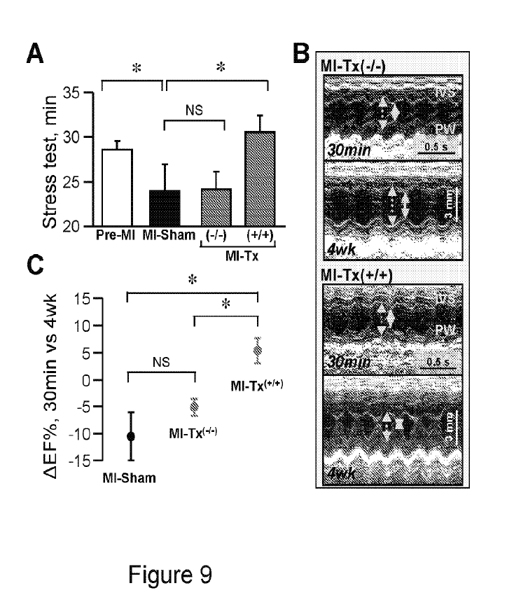

Cardiac performance of the three cohorts compared to pre-infarction condition was first assessed with exercise stress tests. Animals were allowed to run with increasing speed and incline, and distance was recorded. As noted in FIG. 9A, animals treated with CXCR4⁻/Flk-1⁻ out performed both placebo infused as well as animals infused with CXCR4⁻/Flk-1⁻ progenitor cells. CXCR4⁻/Flk-1⁻ treated animals performed as well as pre-infarction animals. Notably, CXCR4⁻/Flk-1⁻ treated animals were statistically similar to placebo infused animals. Echocardiograms at 4-weeks after infarction revealed significant improvement of contractile function and myocardial structure in CXCR4⁺/Flk-1⁺ treated animals but not CXCR4⁻/Flk-1⁻ treated animals (FIG. 9B). Prospective follow-up demonstrated on echocardiography essentially identical ejection fraction between CXCR4⁻/Flk-1⁻ treated animals compared to placebo infused animals. In contrast, CXCR4⁻/Flk-1⁻ treated animals demonstrated significant benefit in cardiac function compared to other cohorts (FIG. 9C).

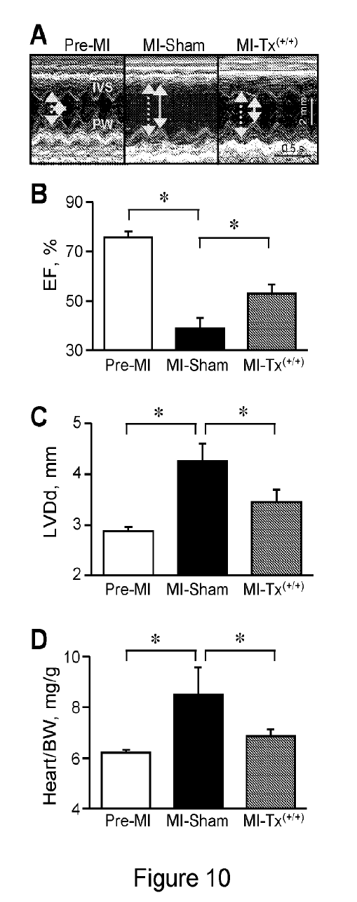

A more detailed comparison of cardiac function using echocardiography in FIG. 10 A-D showed differences in M-mode of the left ventricular ejection fraction (EF) between placebo and CXCR4⁻/Flk-1⁻ treated animals. The left ventricular diastolic dimensions (LVDd) was reduced in CXCR4⁻/Flk-1⁻ treated animals indicating abrogation of maladaptive remodeling associated with ischemic heart disease notable in the placebo cohort. Heart to body weight ratio, a sensitive predictor of prognosis, was improved with CXCR4⁻/Flk-1⁻ treatment compared to placebo infused animals. Thus, intra-venous infusion of CXCR4⁻/Flk-1⁻ cardiac progenitors demonstrates significant therapeutic benefit in myocardial infarction.

### Example 8

### Adult Circulating Blood Contains CXCR4⁻/Flk-1⁻ Progenitor Cells

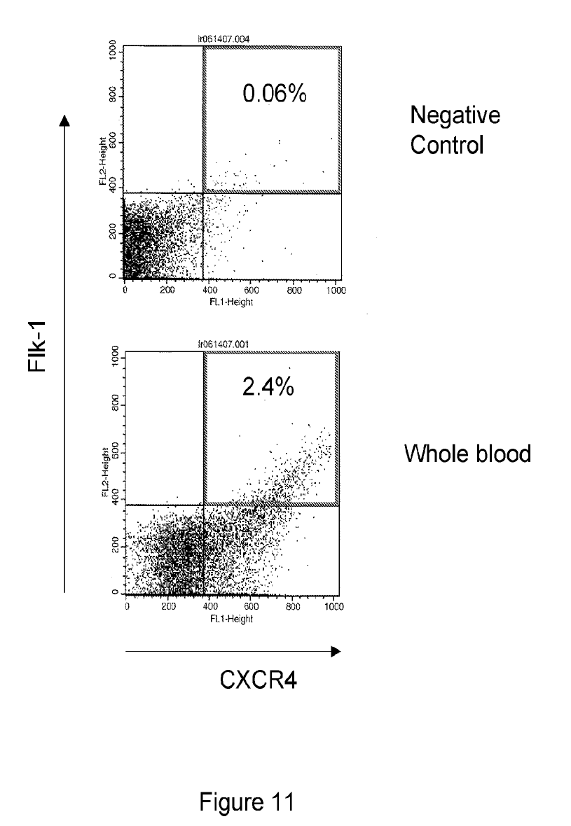

To explore whether CXCR4⁻/Flk-1⁻ progenitor cells are present in circulating blood, whole blood was isolated from adult mice and prepared for FACS analysis using CXCR4 and Flk-1 antibodies. Negative control samples were done to account for non-specific antibody binding. Whole blood contained 2.4% of CXCR4⁻/Flk-1⁻ progenitor cells. See FIG. 11. This demonstrates that in adulthood, a dedicated percentage of circulating cells are CXCR4⁻/Flk-1⁻ progeny amenable to isolation for therapeutic use.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An enriched population of stem cells comprising a CXCR4 polypeptide and an Flk-1 polypeptide, wherein said stem cells are capable of differentiating into cells that express Mef2C, GATA-4, Myocardin, and Nkx2.5 polypeptides.

2. The enriched population of claim 1, wherein said stem cells are human stem cells.

3. The enriched population of claim 1, wherein said stem cells are embryonic stem cells.

4. A method for obtaining cardiac-specific progenitor cells, wherein said method comprises obtaining an enriched population of CXCR4+/Flk-1+ stem cells capable of differentiating into cells that express Mef2C, GATA-4, Myocardin, and Nkx2.5 polypeptides from a population of stem cells.

5. The method of claim 4, wherein said CXCR4+/Flk-1+ stem cells are CXCR4+/Flk-1+ embryonic stem cells.

6. The method of claim 4, wherein said population of stem cells comprises CXCR4+/Flk-1+ stem cells and CXCR4−/Flk-1− stem cells.

7. The method of claim 4, wherein said CXCR4+/Flk-1+ stem cells are human cells.

8. The method of claim 4, wherein said obtaining step comprises using anti-CXCR4 antibodies, anti-Flk-1 antibodies, and a cell sorter to obtain said enriched population of CXCR4+/Flk-1+ stem cells.

9. The method of claim 4, wherein said obtaining step comprises using anti-CXCR4 antibodies, anti-Flk-1 antibodies, and panning to obtain said enriched population of CXCR4+/Flk-1+ stem cells.

10. A method for providing heart tissue with cardiomyocytes, wherein said method comprises administering, to said heart tissue, an enriched population of stem cells comprising a CXCR4 polypeptide and an Flk-1 polypeptide, wherein said stem cells are capable of differentiating into cells that express Mef2C, GATA-4, Myocardin, and Nkx2.5 polypeptides.

11. The method of claim 10, wherein said cardiomyocytes are human cardiomyocytes.

12. The method of claim 10, wherein said stem cells are embryonic stem cells.

13. The method of claim 10, wherein said heart tissue is ischemic heart tissue.

14. The method of claim 10, wherein said heart tissue has suffered from myocardial infarction.

15. A method for providing heart tissue with cardiomyocytes, wherein said method comprises administering, to said heart tissue, cardiomyocytes obtained from an enriched population of stem cells comprising a CXCR4 polypeptide and an Flk-1 polypeptide, wherein said stem cells are capable of differentiating into cells that express Mef2C, GATA-4, Myocardin, and Nkx2.5 polypeptides.

16. The method of claim 15, wherein said cardiomyocytes are human cardiomyocytes.

17. The method of claim 15, wherein said stem cells are embryonic stem cells.
